(19) United States
(12) Patent Application Publication
(10) Pub. No.: US 2025/0082311 A1
(43) Pub. Date: Mar. 13, 2025

(54) TISSUE FRAGMENTER AND METHODS OF USING THE SAME

(71) Applicant: IOVANCE BIOTHERAPEUTICS, INC, San Carlos, CA (US)

(72) Inventors: Joseph James Wypych, Tracy, CA (US); Richard Boyt Love, Berkeley, CA (US); Neil Foxman, San Diego, CA (US); Minh Duong, San Diego, CA (US)

(21) Appl. No.: 18/728,574

(22) PCT Filed: Jan. 13, 2023
(86) PCT No.: PCT/US2023/060668

§ 371 Date: Jul. 12, 2024

Related U.S. Application Data
(60) Provisional application No. 63/299,346, filed on Jan. 13, 2022.

(51) Int. Cl. A61B 10/02; G01N 1/06

(52) U.S. Cl. CPC A61B 10/02; G01N 2001/065

(57) ABSTRACT

A tissue fragmenting device and methods of using the same are provided herein. A device for preparing a plurality of tissue fragments from a tissue sample includes a platform having a central opening, a shuttle slidably coupled to the platform and including a shuttle blade, a blade array mounted across the central opening of the platform having a plurality of blades each generally perpendicular to the shuttle blade, and a piston slidably coupled to the platform and slidably coupled to the shuttle along a predefined raceway and configured to urge the tissue sample into the blade array.

### CROSS REFERENCE TO RELATED APPLICATIONS

This application claims the benefit of U.S. Provisional Application Ser. No. 63/299,346, filed Jan. 13, 2022, which is incorporated herein by reference in its entirety.

### FIELD OF THE DISCLOSURE

The disclosure described herein relates generally to a device for reducing tissue samples to fragments and more particularly, but not exclusively, to a plurality of uniform fragments.

### BACKGROUND OF THE DISCLOSURE

A need in the field exists for dividing or fragmenting tissue samples of varied composition, density, and properties into uniform fragments for use in biochemical assays and procedures.

### SUMMARY OF THE DISCLOSURE

The devices provided herein meet the needs in the field by providing tissue fragmentation devices capable of cutting tissue samples in at least two axes to provide uniform tissue fragments.

In an embodiment, the disclosure includes a device for preparing a plurality of tissue fragments from a tissue sample. In some embodiments, the tissue sample is a tumor sample obtained from a subject (e.g., a human or animal subject). In some embodiments, the device includes a platform having a central opening. In some embodiments, the device includes a shuttle slidably coupled to the platform and including a shuttle blade. In some embodiments, the device includes a blade array mounted across the central opening of the platform and comprising a plurality of blades each generally perpendicular to the shuttle blade. In some embodiments, the device includes a piston slidably coupled to the platform and slidably coupled to the shuttle along a predefined raceway and configured to urge the tissue sample into the blade array. In some embodiments, moving the shuttle through the raceway moves the shuttle blade and the blade array through a bottom portion of the tissue sample to cut the plurality of tissue fragments from the tissue sample.

In some embodiments, the platform comprises a channel and the shuttle is slidably coupled to the platform through the channel.

In some embodiments, the raceway comprises a plurality of races. In some embodiments, each race of the plurality of races comprises an increment ramp. In some embodiments, each race of the plurality of races is separated by about 0.5 mm to about 10 mm. In some embodiments, each race of the plurality of races is separated about 1 mm to about 5 mm. In some embodiments, each race of the plurality of races is separated by about 3 mm. In some embodiments, each race of the plurality of races is separated by about 1 mm.

In some embodiments, each tissue fragment that may be prepared by the device described herein may be of uniform size. In some embodiments, the plurality of tissue fragments may be uniform. In some embodiments, the plurality of tissue fragments have a thickness of about 0.5 mm to about 10 mm. In some embodiments, the plurality of tissue fragments have a thickness of about 1 mm to about 5 mm. In some embodiments, the plurality of tissue fragments have a thickness of about 3 mm. In some embodiments, the plurality of tissue fragments have a thickness of about 1 mm.

In some embodiments, the shuttle may comprise a guide pin that may be connected to the raceway. In some embodiments, the shuttle comprises at least one guide pin that is slidably connected to the raceway.

In some embodiments, moving the shuttle through the raceway moves the shuttle blade and then the blade array through a bottom portion of the tissue sample. In some embodiments, moving the shuttle through the raceway moves the blade array and the shuttle blade through a bottom portion of the tissue sample.

In some embodiments, the devices described herein may include a motor configured to move the shuttle through the raceway.

In some embodiments, the raceway comprises at least two raceways.

In an embodiment, the disclosure includes a method for using a device described herein for cutting a tissue sample from a patient into a plurality of fragments. In some embodiments, the disclosure includes a method of using a device described herein comprising the step of loading the tissue sample into the tissue sample mount and moving the shuttle through the raceway to cut the tissue sample in two axes to prepare a plurality of tissue fragments from the tissue sample.

In some embodiments, each tissue fragment that may be prepared by the methods described herein may be of uniform size. In some embodiments, the plurality of tissue fragments may be uniform. In some embodiments, the plurality of tissue fragments have a thickness of about 0.5 mm to about 10 mm. In some embodiments, the plurality of tissue fragments have a thickness of about 1 mm to about 5 mm. In some embodiments, the plurality of tissue fragments have a thickness of about 1 mm to about 4 mm. In some embodiments, the plurality of tissue fragments have a thickness of about 1 mm to about 3 mm. In some embodiments, the plurality of tissue fragments have a thickness of about 1 mm to about 2 mm. In some embodiments, the plurality of tissue fragments have a thickness of about 1 mm. In some embodiments, the plurality of tissue fragments have a thickness of about 0.5 mm to about 10 mm. In some embodiments, the plurality of tissue fragments have a thickness of about 0.1 mm to about 0.5 mm. In some embodiments, the plurality of tissue fragments have a thickness of about 0.5 mm to 3 mm. In some embodiments, the plurality of tissue fragments have a thickness of about 0.5 mm to 2.5 mm. In some embodiments, the plurality of tissue fragments have a thickness of about 0.5 mm to 2 mm. In some embodiments, the plurality of tissue fragments have a thickness of about 0.5 mm to 1.5 mm.

In some embodiments, provided herein are methods of using the device as described herein to cut a tissue sample from a patient into a plurality of fragments, the method comprising loading the tissue sample into the tissue sample mount and moving the shuttle through the raceway to cut the tissue sample in two axes to prepare a plurality of tissue fragments from the tissue sample. In some embodiments, the plurality of tissue fragments each have a thickness of about 0.5 mm to about 10 mm. In some embodiments, the plurality of tissue fragments each have a thickness of about 1 mm to about 5 mm. In some embodiments, the plurality of tissue fragments each have a thickness of about 3 mm. In some embodiments, the plurality of tissue fragments each have a thickness of about 1 mm. In some embodiments, the plurality of tissue fragments each have a width of about 0.5 mm to about 10 mm. In some embodiments, the plurality of tissue fragments each have a width of about 1 mm to about 5 mm. In some embodiments, the plurality of tissue fragments each have a width of about 3 mm. In some embodiments, the plurality of tissue fragments each have a width of about 1 mm.

### BRIEF DESCRIPTION OF THE DRAWINGS

The foregoing summary, as well as the following detailed description of the disclosure, will be better understood when read in conjunction with the appended drawings.

FIG. 1 is a front perspective view of a device for preparing a plurality of tissue fragments in accordance with an exemplary embodiment of the present disclosure shown in an open configuration;

FIG. 2 is a rear perspective view of the device of FIG. 1;

FIGS. 3A-1, 3A-2, 3B-1, and 3B-2 are side elevational views of the device of FIG. 1;

FIGS. 4A-4B illustrate the motion of the device making a horizontal cut;

FIG. 5A-5B are perspective views of a shuttle of the device of FIG. 1;

FIG. 6 is a perspective view of a piston of the device of FIG. 1;

FIGS. 7A-7B are perspective views of a platform of the device of FIG. 1;

FIGS. 8A-8H are top plan views of the shuttle blade according to some embodiments illustrating example blade shapes for use with the device of FIG. 1; and

FIGS. 9A-9C are enlarged views of the guides and ribs of the device of FIG. 1.

### DETAILED DESCRIPTION OF THE DISCLOSURE

A number of assays are known in the art that may require tissue fragments or pieces of uniform and consistent size. For example, adoptive cell therapy (ACT) relies on the use of tumor fragments or pieces for the culturing and expansion of endogenous tumor reactive T-cells (e.g., Tumor Infiltrating Lymphocytes), which may then be delivered to cancer patients. However, animal tissue, and especially cancerous animal tissue, does not have a uniform density or texture and provides a challenge when attempting to reduce such tissue to uniform fragments or pieces. As used herein, the term “tissue” refers to animal tissue. In some embodiments, the tissue may be tumor tissue. In some embodiments, a tissue sample may be a solid tumor or a portion thereof that may have been surgically excised from a patient. In some embodiments, a tissue sample may be tumor biopsy sample.

To meet the needs in the field, devices of the disclosure allow for the fragmentation and cutting of a tissue sample into a number of uniform fragments or pieces by cutting the sample in multiple axes.

In some embodiments, a tissue sample that may be fragmented by device 1 may have a width of less than 0.5, 0.6, 0.7, 0.8, 0.9, 1, 2, 3, 4, 5, 6, 7, 8, 9, 10, 11, 12, 13, 14, 15, 16, 17, 18, 19, or 20 mm. In some embodiments, a tissue sample that may be fragmented by device 1 may have a width of greater than 0.5, 0.6, 0.7, 0.8, 0.9, 1, 2, 3, 4, 5, 6, 7, 8, 9, 10, 11, 12, 13, 14, 15, 16, 17, 18, 19, or 20 mm. In some embodiments, a tissue sample that may be fragmented by device 1 may have a width of about 0.5, 0.6, 0.7, 0.8, 0.9, 1, 2, 3, 4, 5, 6, 7, 8, 9, 10, 11, 12, 13, 14, 15, 16, 17, 18, 19, or 20 mm.

In some embodiments, a tissue sample that may be fragmented by device 1 may have a width of less than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, a tissue sample that may be fragmented by device 1 may have a width of greater than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, a tissue sample that may be fragmented by device 1 may have a width of about 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm.

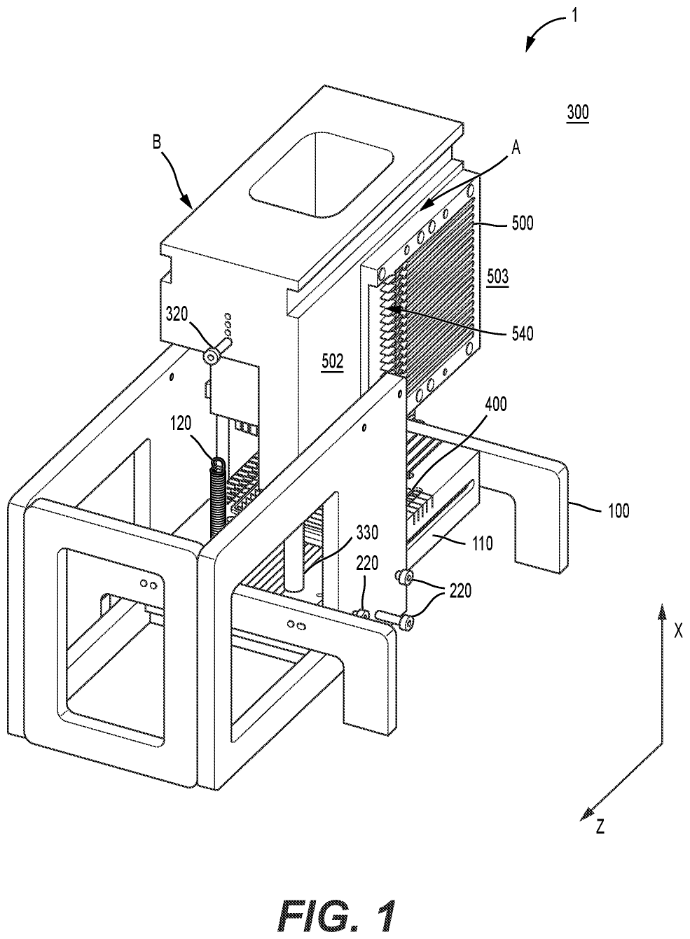

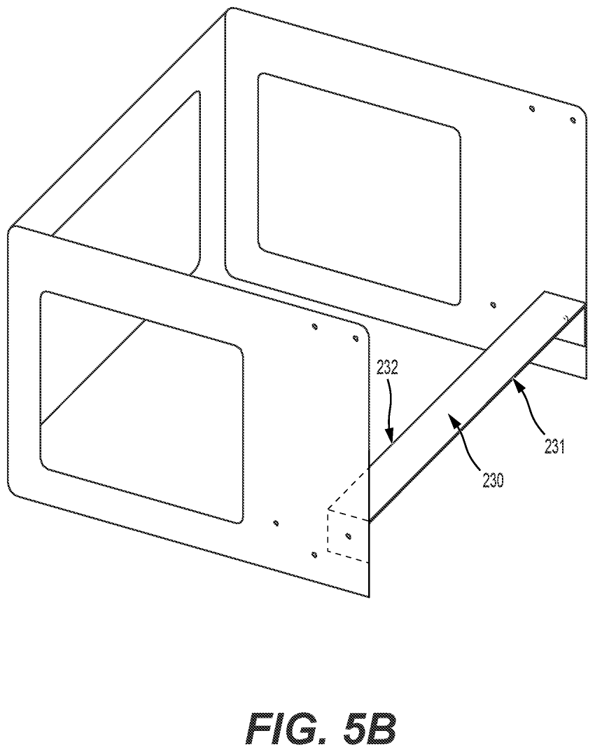

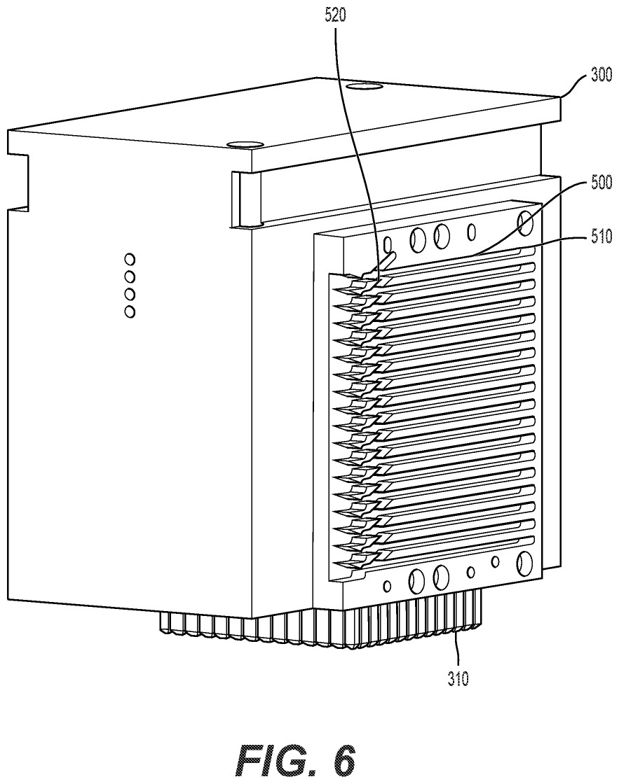

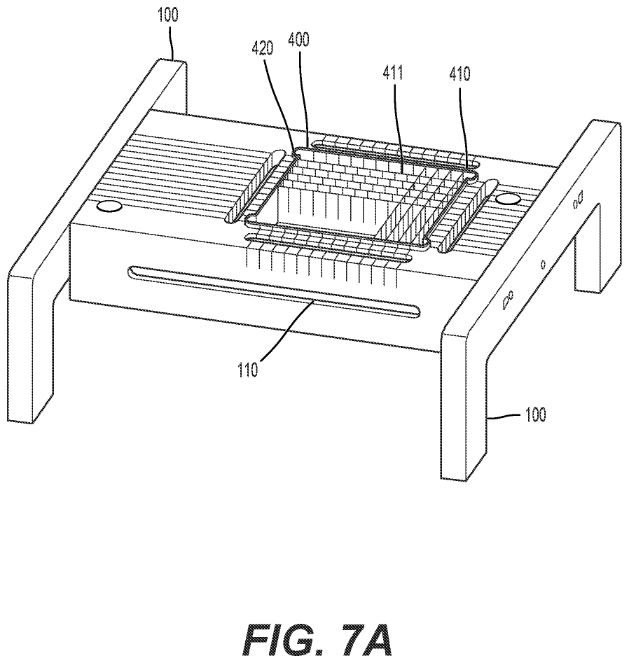

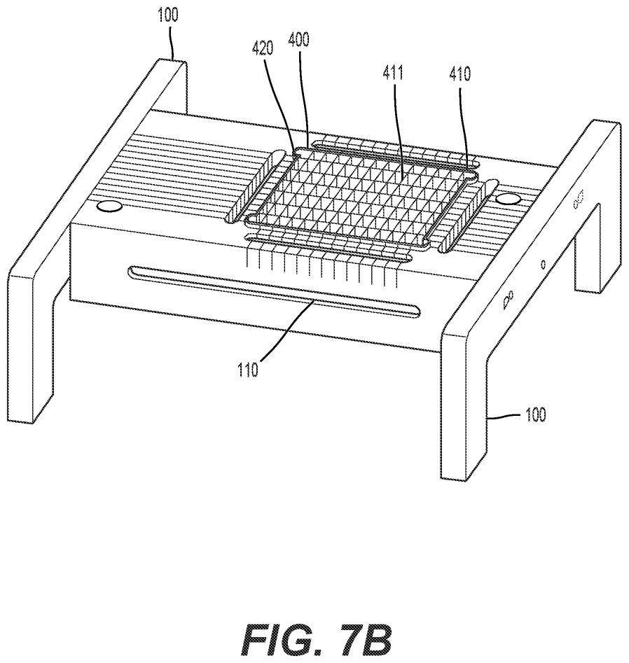

Referring now to the figures, wherein like elements are numbered alike throughout, FIGS. 1-9 refer to a device 1 for cutting and fragmenting tissue to provide a plurality of tissue fragments.

FIGS. 1-3 provide various views of a device 1 that may be used to fragment and cut tissue and thereby provide a plurality of tissue fragments or pieces. In an embodiment, the device 1 includes a platform 100, and shuttle 200, and a piston 300. As shown in FIG. 1, the platform 100, shuttle 200, and piston 400 are movably coupled to one another as described herein.

Platform 100 may include a blade array 400 and may be coupled to the shuttle 200 via channels 110, which may be disposed on either side of the platform 100. In some embodiments, platform 100 may be movably coupled to shuttle 200 through the channels 110. In some embodiments, platform 100 includes one channel 110. In some embodiments, platform 100 includes at least two channels 110.

In some embodiments, shuttle 200 includes one or more guide pin(s) 220 that register with channels 110 and allow for the shuttle 200 to track in a predetermined sliding configuration relative to the platform 100. For example, the shuttle 200 may be configured to slide horizontally with respect to the platform 100 as guide pin(s) 220 track with the channels 110.

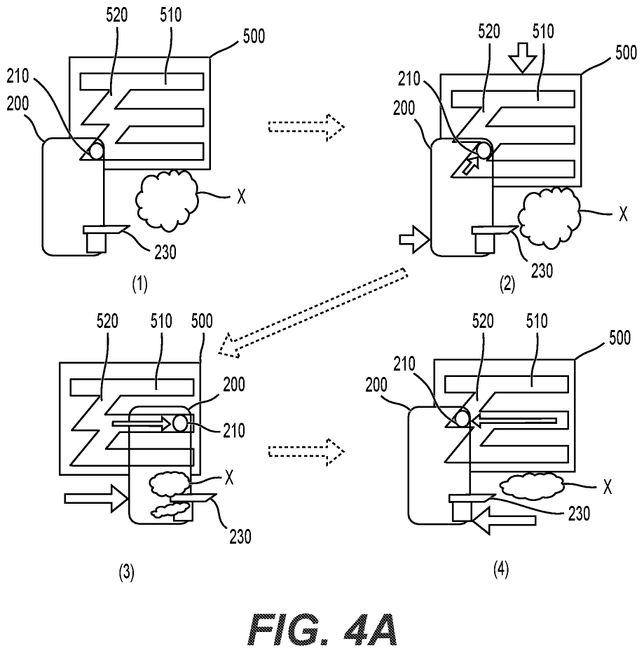

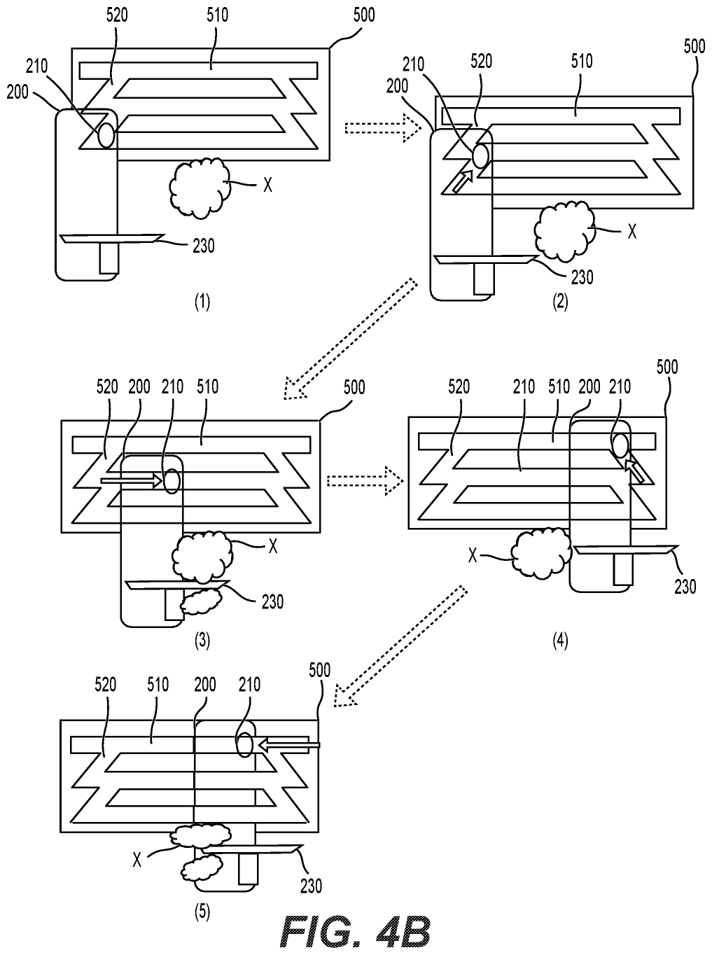

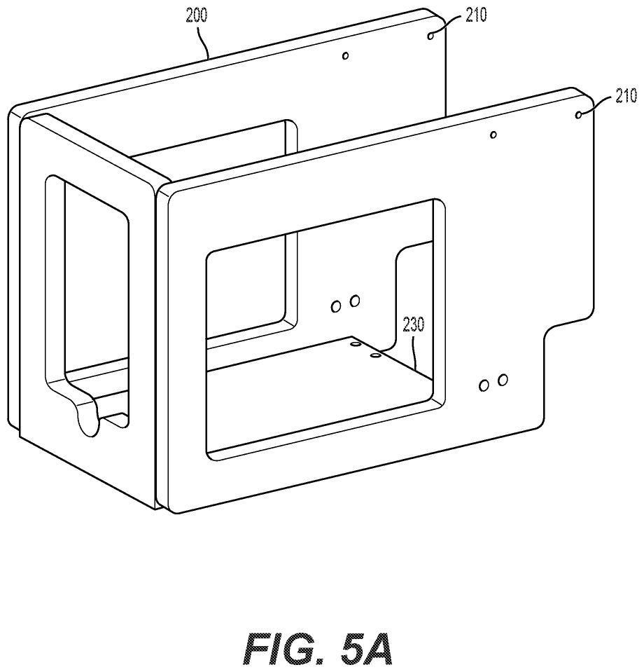

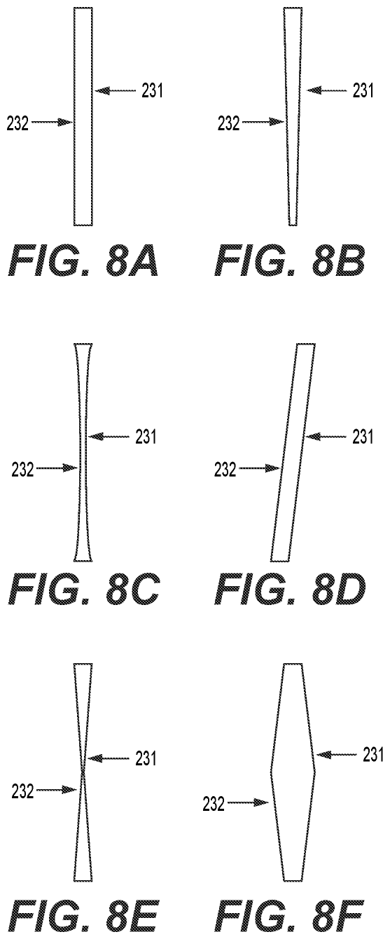

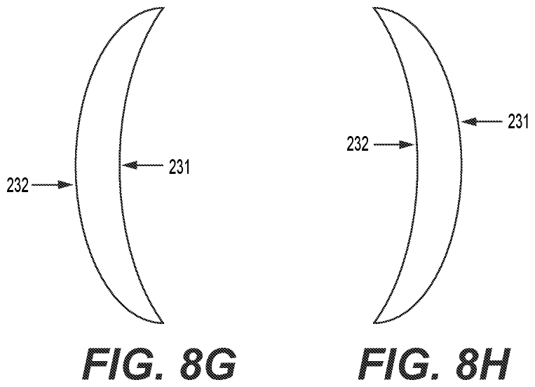

Shuttle 200 may also include a blade 230 (also referred to herein as “shuttle blade” 230) configured to cut a tissue sample along a horizontal axis (i.e., a Z-axis) (see FIGS. 4A-4B). Blade 230, for example, may be a ceramic blade or a metal blade (e.g., stainless steel). FIGS. 8A-8H illustrate possible blade shapes of some embodiments. In some embodiments, blade 230 includes a single sharpened edge (“cutting edge”) configured to cut through the tissue sample X. In some embodiments, blade 230 includes at least one cutting edge. In other embodiments, as shown in FIG. 5B, the shuttle blade 230 may have two edges, a first edge 231 and a second edge 232. In some embodiments, both first edge 231 and second edge 232 are configured as cutting edges, e.g., sharp enough to cut through the tissue sample. Edges 231 and 232 may be positioned on opposite sides of blade 230. In some such embodiments, for example, having two cutting edges allows blade 230 to cut the tissue sample from either direction as blade 230 is moved back and forth with respect to the tissue sample, as will be described further herein.

In some embodiments, the blade 230 has a rectangular shape (FIG. 8A) or has a parallelogram shape (FIG. 8D), for example, such that first edge 231 and second edge 232 are straight and substantially parallel. In some embodiments, the blade 230 has a triangular shape, trapezoidal shaped, or is angled (FIG. 8B). In some such embodiments, first edge 231 and second edge 232 may be obliquely angled with respect to each other. In some embodiments, blade 230 has a polygonal shape, for example, a concave polygonal shape (e.g., FIG. 8E) or a convex polygonal shape (e.g., FIG. 8F). In some such embodiments, rather than having a straight linear configuration, edges 231 and 232 may each have one or more angles. In some embodiments, one or more edges of the blade 230 has a curved shape (e.g., FIG. 8C). In some embodiments, the blade 230 has a crescent shape (FIGS. 8G and 8H). As noted above, in some embodiments, the shuttle blade 230 is double-edged, for example, where each of edges 231 and 232 is sharpened for slicing through the tissue sample. In some embodiments, the shuttle blade 230 is double-edged, and each edge is angled or has a triangular shape (FIG. 8E or 8F). In some embodiments, the shuttle blade 230 is double-edged, and each edge is rectangular or is straight (FIG. 8A, 8D). In some embodiments, the shuttle blade 230 is double-edged, and each edge has a curved shape (FIG. 8C). In some embodiments, the shuttle blade 230 is double-edged, and each edge has a crescent shape (FIG. 8G, FIG. 8H).

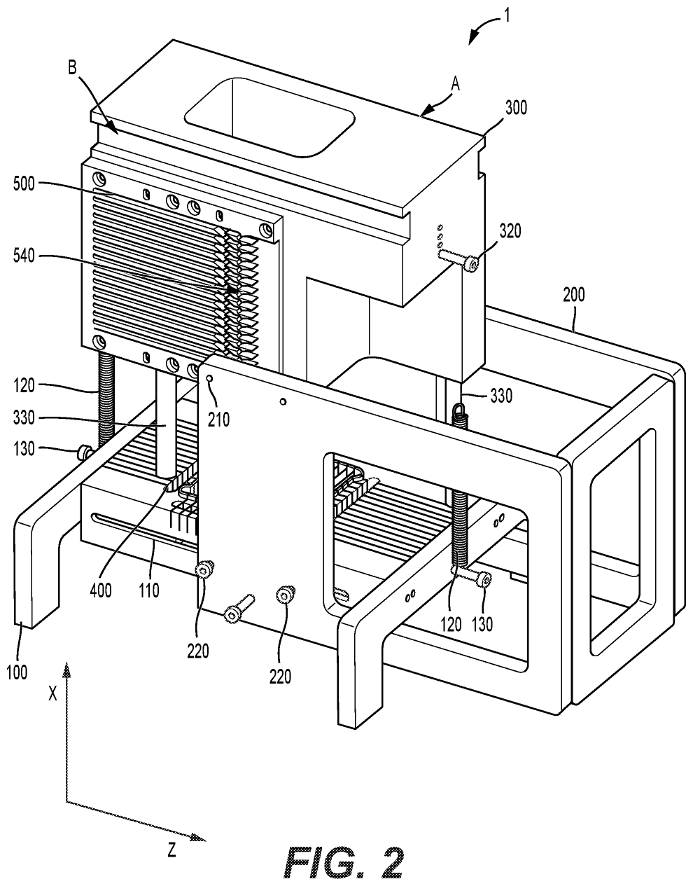

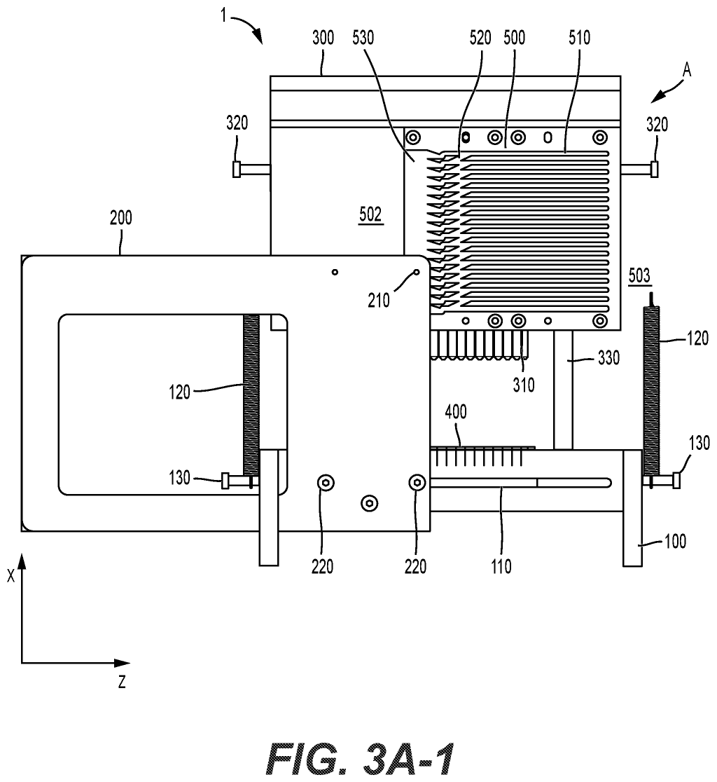

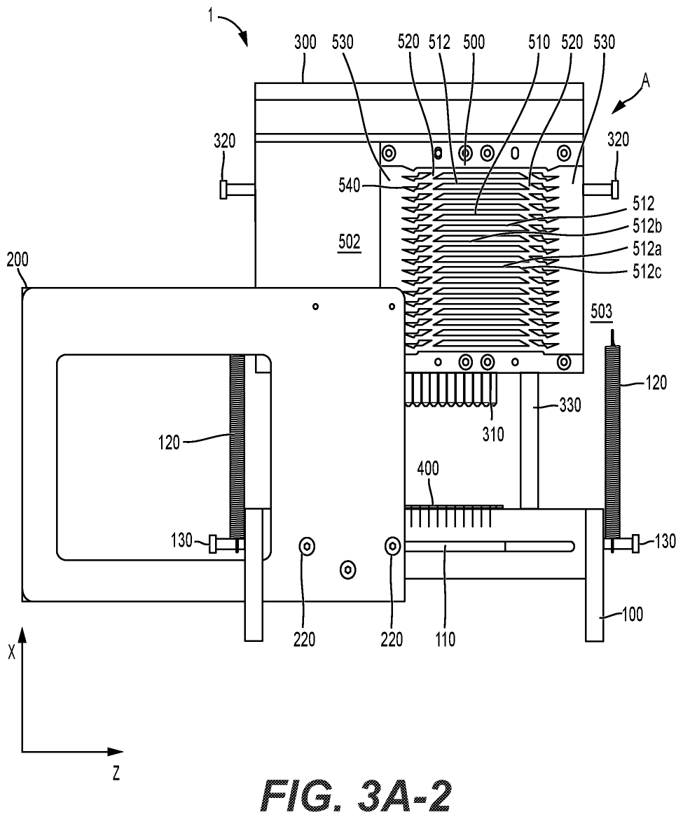

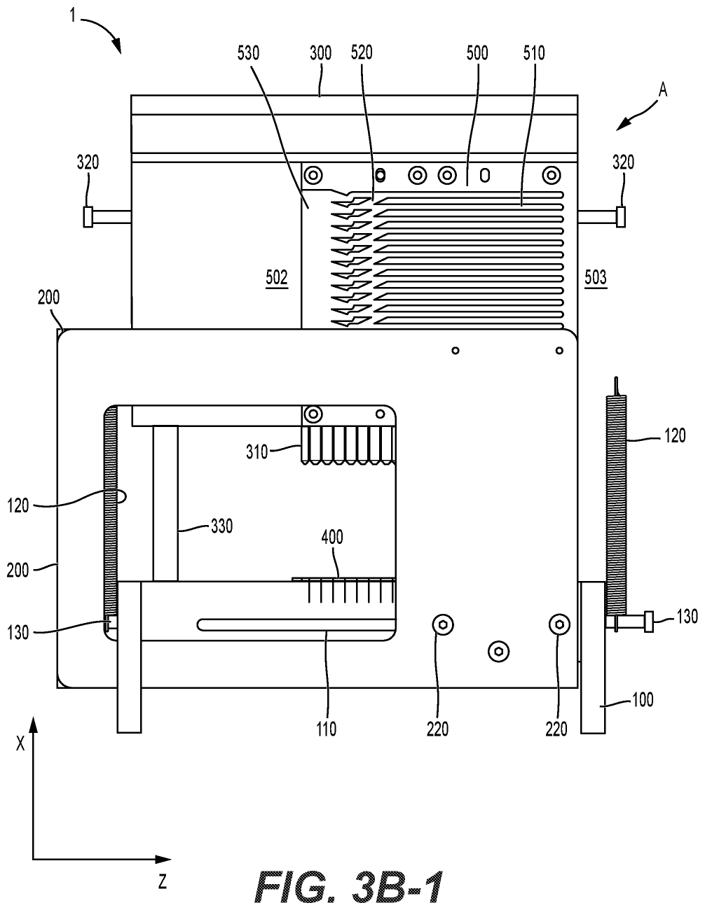

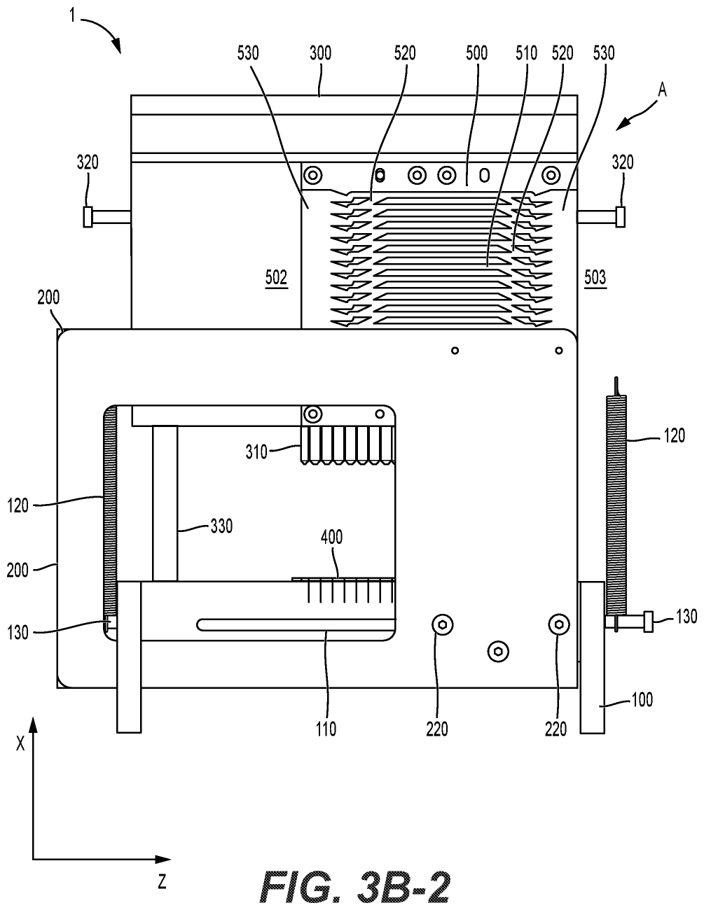

FIGS. 1, 2, 3A-1, and 3A-2 illustrate piston 300 with a tissue mount 310 and a raceway 500. Raceway 500 may have a first end 502 and a second end 503. Raceway 500 may include a plurality of races 510. Races 510 may be disposed in a substantially parallel configuration on one or more sides of piston 300. Races 510 may be defined by longitudinal ribs 512. Longitudinal ribs 512 may be positioned above and below each race 510, for example. FIG. 3A-2 is illustrated with seventeen longitudinal ribs. As illustrated in FIG. 3A-2, ribs 512 may include a top longitudinal face 512a and a bottom longitudinal face 512b which may be generally parallel to each other. In some embodiments, top longitudinal face 512a is shorter than bottom longitudinal face 512b. For example, ribs 512 may include a ramped face 512c on one or two lateral ends of rib 512. Ramped face 512c maybe disposed between top longitudinal face 512a and bottom longitudinal face 512b. Ramped face 512c, in some embodiments, extends from top longitudinal face 512a to bottom longitudinal face 512b and may be obliquely angled with respect to top longitudinal face 512a and acutely angled with respect to bottom longitudinal face 512b. Advantages of ramped face 512c includes facilitating a smooth transition of pin 210 into race 510 from one or both ends of race 510.

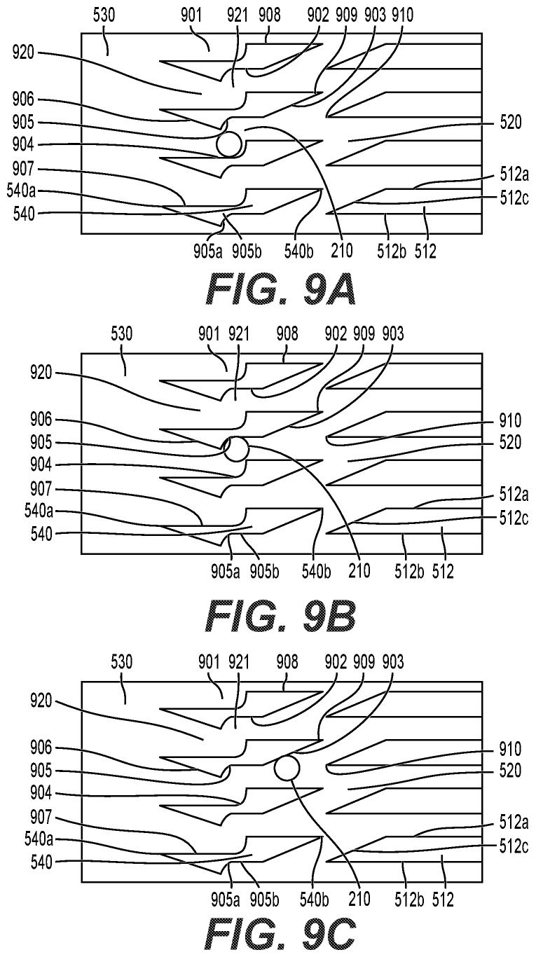

In some embodiments, piston 300 also includes guide(s) 540. In some embodiments, piston 300 includes one or more sets of guides 540. For example, in the embodiment shown in FIG. 3A-1, there is a first set of guides 540 that are separate from and positioned between longitudinal ribs 512 and the first end 502 of raceway 500. In the embodiment shown in FIG. 3A-2, a second set of guides 540 may be present and positioned between longitudinal ribs and second end 503 of raceway 500. In some embodiments, each set of guides 540 includes a plurality of guides 540 that may be vertically aligned. In some embodiments, the number of guides 540 included in each set may be equal to the number of longitudinal ribs 512. Guides 540, in some embodiments, may be disposed in horizontal alignment with ribs 512. In some embodiments, as illustrated in FIGS. 9A-9C, guide(s) 540 have an upper surface 901 and a lower surface 902. The upper and lower surfaces 901 and 902 can be configured to engage guide pin(s) 210 as described in more detail below. In some embodiments, a pair of adjacent guides 540 are separated by a pinway 920 that is defined between the lower surface 902 of a first guide 540 of the pair and the upper surface 901 of the second guide 540 of the pair. In the illustrated embodiment, upper surface 901 includes two longitudinal edges, a first longitudinal edge 907 and a second longitudinal edge 908. First longitudinal edge 907 may be parallel (or substantially parallel) to second longitudinal edge 908. For example, first longitudinal edge 907 and second longitudinal edge 908 may be oriented substantially horizontally when piston 300 is in operation. In some embodiments, in each guide 540 the second longitudinal edge 908 may be vertically offset from first longitudinal edge 907. For example, the second longitudinal edge 908 may be positioned higher than first longitudinal edge 907.

Upper surface 901 may also include an upper connector 904. In some embodiments, upper connector 904 is interposed between first longitudinal edge 907 and second longitudinal edge 908. In some embodiments, upper connector 904 may include a curved face (e.g., a convexly curved face). The curved face of upper connector 904 may be defined by at least one radius. In some embodiments, upper connector 904 has a curvature with a radius that approximates the radius of the guide pin(s) 210. In some embodiments, the curvature of upper connector 904 is defined by a convex circular arc that extends from first longitudinal edge 907 to second longitudinal edge 908.

The lower surface 902 of each guide 540 may have two or more planar edges. For example, lower surface 902 may include a first lower planar edge 903 and a second lower planer edge 906. First lower planar edge 903 and second lower planar edge 906 may be non-parallel with respect to one another. In some embodiments, first lower planar edge 903 and second lower planar edge 906 may be configured such that respective projections of first lower planar edge 903 and second lower planar edge 906 intersect within adjacent pinway 920 below lower surface 902. The first lower planar edge 903 may be disposed at an acute angle with respect to the second longitudinal edge 908 of the upper surface 901. The second lower planar edge 906 may be angled acutely in the downward direction with respect to the first longitudinal edge 907 of the upper surface 901. Guide 540 may have an interior end 540b and an exterior end 540a. Interior end 540b may have a pointed configuration defined by the intersection of second longitudinal edge 908 and first lower planar edge 903 which may intersect to form an acute angle. Exterior end 540a may have a pointed configuration defined by the intersection of first longitudinal edge 907 and second lower planar edge 906 which may intersect to form an acute angle.

First lower planar edge 903 and second lower planar edge 906 can be connected by a lower connector 905. In some embodiments, lower connector 905 includes a curved face 905a (e.g., a convexly curved face) and a horizontal face 905b. The curved face 905a of lower connector 905 may be defined by at least one radius. In some embodiments, curved face 905a of lower connector 905 has a curvature with a radius that approximates the radius of the guide pin(s) 210. In some embodiments, the radius of curvature of curved face 905a is the same as the radius of curvature of the curved face of upper connector 904. In some embodiments, the curvature of curved face 905a of lower connector 905 is defined by a a convex circular arc that extends from the second lower planar edge 906 to the horizontal face 905b. The horizontal face 905b extends from the curved face 905a to the first lower planar edge 903.

In one embodiment, piston 300 includes a plurality of guides 540 that are configured to form a plurality of pinways 920 disposed between adjacent guides 540. Each pinway 920 may be configured to form a channel to direct the guide pin 210. For example, pinway 920 may be configured to direct a guide pin 210 from outer region 530 on the outer limit of the raceway 500 towards races 510 or direct the guide pin 210 from races 510 to outer region 530. In some embodiments, pinway 920 provides a pathway in the vertical direction (the X-axis) for movement of piston 300 as the guide pin 210 traces a path relative to raceway 500 through pinway 920, as defined by two adjacent guides 540. The pinway(s) 920 may comprise a vertical subchannel 921 having features that are configured to arrest the motion of the guide pin 210 in the horizontal direction (the Z-axis). For example, if force is exerted upwards in the vertical direction (along the X-axis) on piston 300 so as to keep the guide pin 210 in contact with an upper connector 904 of one guide 540, when such force is released the pinway(s) 920 may direct piston 300 downwards in the vertical direction (along the X-axis), causing the guide pin 210 to disengage from the upper connector 904 of said one guide 540 and to engage a lower connector 905 of an adjacent (higher) guide 540. In another example, if force is exerted downwards in the vertical direction (along the X-axis) on piston 300 so as to keep the guide pin 210 in contact with a lower connector 905 of one guide 540, when such force is released the pinway(s) 920 may direct piston 300 upwards in the vertical direction (along the X-axis), causing the guide pin 210 to disengage from the lower connector 905 of said one guide 540 and to engage an upper connector 904 of an adjacent (lower) guide 540 as described in further detail below.

As further shown in FIGS. 9A-9C, pinway(s) 920 are configured to direct the guide pin(s) from outer region 530 on the outer limit of the raceway 500 towards races 510 or direct the guide pin(s) 210 from races 510 to outer region 530 when shuttle 200 is moving in a horizontal direction (along the Z-axis). For example, as biasing member 120 (e.g., one or more springs) is biasing piston 300 downwards (along the X-axis), guide 540 is configured to direct the elevation of the piston 300 as shuttle 200 is moving horizontally. The pinway(s) 920 are defined in part by the lower surface 902 of a guide 540 and the upper surface 901 of an adjacent guide 540 and can be configured to guide the movement of piston 300 in the vertical direction (along the X-axis). The pinway(s) 920 can extend from the outer limits of the interior end 540b of the guide 540 to the outer limits of the exterior end 540a of a guide 540. As the guide pin 210 moves laterally towards the raceway 500, the guides 540 are configured to arrest the vertical motion of the piston 300 between the upper surfaces 901 and lower surfaces 902 of the guides 540.

The guide pin 210 may move from outer region 530 towards the races 510. For example, guide pin(s) 210 may interact with a second lower planar edge 906 of the lower surface 902 of an upper guide 540 or a first longitudinal edge 907 of the upper surface 901 of a lower guide 540. The guide pin 210 can then move through the pinway 920 until guidepin(s) 210 reach the upper connector 904 (as shown in FIG. 9A) of the lower guide 540. Once the guide pin(s) 210 reach the upper connector 904 of the lower guide 540, the downward force of biasing member 120 on piston 300 will direct the lower connector 905 of the lower surface 902 of the upper guide 540 (as shown in FIG. 9B) towards the guide pin(s) 210. The lower connector 905 of the upper guide 540 is configured to restrict movement of piston 300 in the downwards direction (along the X-axis) and restrict the movement of shuttle 200 outwards in the horizontal direction (along the Z-axis) towards the outer region 530 while the guide pin 210 is engaged with the surface of lower connector 905. When the shuttle 200 is moved in the horizontal direction (along the Z-axis) inwards towards the races 510, the guide pin 210 will trace a path relative to raceway 500 through pinway 920 towards the increment ramp 520 along a first lower planar edge 903 on the lower surface 902 of the upper guide 540 (FIG. 9C) and then into races 510.

The guide pin(s) 210 may move from the races 510 towards the outer region 530 on the outer limit of the raceway 500. For example, the guide pin(s) 210 may first interact with either the first lower planar edge 903 of the upper guide 540 or the second longitudinal edge 908 of the lower guide 540 (FIG. 9C). The guide pin(s) 210 may then trace a path relative to raceway 500 through the pinway 920 towards the lower connector 905 of the upper guide 540. The downward force of biasing member 120 on piston 300 will hold the guide pin 210 at the lower connector 905 of the upper guide 540 (FIG. 9B) until the user applies an upward force on the piston 300 (e.g., opposite the force of biasing member 120). When a user moves the shuttle 200 horizontally (along the Z-axis) towards the outer region 530 and simultaneously applies an upward force on the piston 300 that is greater than the downward force of biasing member 120 while the guide pin(s) 210 is at the lower connector 905 of the upper guide 540, the guide pin(s) 210 will disengage from lower connector 905 of the upper guide, the piston 300 will move upwards vertically (along the X-axis) and cause the guide pin(s) 210 to engage the upper connector 904 of the lower guide (FIG. 9A) 540. The guide pin 210 can then trace a path relative to raceway 500 out of the pinway 920 into the outer region 530 absent of guides 540 and ribs 512, either along the second lower planar edge 906 of the top guide 540 or the first longitudinal edge 907 of the bottom guide 540 by the horizontal movement of shuttle 200.

As shown in FIG. 3A-2 or 3B-2, in one embodiment, there is a second set of guides 540 disposed on the distal end of the raceway 500. The guides 540 on the distal end of the raceway 500 are arranged in a mirrored configuration to the arrangement described above.

In some embodiments, the tip 909 created by the first lower planar edge 903 and a second longitudinal edge 908 of guide 540 overlaps the tip 910 created by the bottom longitudinal face 512b and the ramped face 512c of rib 512. This overlap may prevent the piston 300 from moving either up or down vertically (in the X axis) unless the shuttle 200 is moved in the horizontal direction (in the Z axis). In some such embodiments, the guide pin(s) 210 are prevented from moving in a manner that would trace a straight path relative to raceway 500 in the space between guides 540 and ribs 512 but allowed to move in a manner that traces a zig-zag pattern relative to raceway 500 through the increment ramps 520.

In some embodiments, piston 300 may include opposing sides (e.g., front side A and back side B). A raceway 500 may be disposed on either or both of the parallel sides. The raceway 500 on the A side and the B side of the piston 300 may be symmetrically aligned and function in the same way. In one embodiment, including raceway 500 on both sides promotes a smooth sliding motion of shuttle 200 guided by one or more guide pin(s) 210 that are registered with corresponding races on opposing sides of piston 300.

In some embodiments, tissue mount 310 is configured to contact the tissue sample and push the tissue sample towards and through blade array 400 of platform 100 as shuttle 200 is moved back and forth in a lateral direction along the Z-axis and guide pin 210 traces a zig-zag path relative to raceway 500 upwards through adjoining races 510 to allow the movement of piston 300 downwards in a vertical direction (along the X-axis). Shuttle 200 may be coupled to the piston 300 through the raceway 500. Shuttle 200 may include one or more guide pin(s) 210 that may register with the races 510 of the raceway 500 such that the guide pin(s) 210 track within the races 510 when the shuttle 200 is moved horizontally in the Z-axis. The shuttle blade 230 may, therefore, make horizontal (i.e., along the Z-axis) cuts in the tissue sample X after the tissue sample has passed through blade array 400.

Piston 300 may be biased toward platform 100 through the use of one or more biasing members 120, which may attach to biasing member mounts 130 and 320. In some embodiments, the biasing member 120 may be one or more springs (e.g., coil springs) or actuators that may bias the piston 300 toward the platform 100. In some embodiments, the biasing members 120 may be springs. In some embodiments, the springs may be connected to biasing member mounts on the platform 100 (i.e., biasing member mounts 130) and the piston 300 (i.e., biasing member mounts 320). As shown in device 1, the biasing members 120 provide a biasing force that pulls the piston 300 toward the platform 100, allowing for blade array 400 to make vertical cuts (i.e., the X-axis) in the tissue sample X as piston 300 (e.g., tissue mount 310) pushes the tissue sample through blade array 400. Piston 300 may be positioned with respect to platform 100 by one or more guides 330, which may orient the tissue mount 310 over the blade array 400.

For embodiments shown in FIGS. 3A-1 and 3B-1, raceway 500 includes races or channels 510 that communicate with shuttle 200 via guide pin(s) 210. Races 510 may also include increment ramps 520. Communication between guide pin(s) 210, races 510, and increment ramps 520 allow for the piston 300 to progress toward the platform 100 when the shuttle 200 slides back and forth horizontally along the Z-axis. For embodiments shown in FIGS. 3A-1 and 3B-1, the relationship between shuttle 200 and raceway 500 is depicted in greater detail in FIG. 4A when slicing a tissue sample X. For embodiments shown in FIGS. 3A-1 and 3B-1, the raceway 500 includes an outer region 530 absent of guides 540 and ribs 512 on the end 502 of the raceway 500. In some embodiments, the outer region 530 could be positioned on end 503 of raceway 500. This outer region 530 absent of guides 540 and ribs 512 allows for the piston 300 to be moved up and down to a desired position quickly, without moving the guide pin(s) 210 through all the races 510 and increments ramps 520. When the guide pin(s) 210 are moved into outer region 530 absent of guides 540 and ribs 512, they are unbound and the piston 300 is free to move in the vertical direction (i.e., the X-axis).

For embodiments shown in FIGS. 3A-1 and 3B-1, the shuttle 200 may be moved through four positions (i.e., positions, 1-4) in the raceway 500, as shown in FIG. 4A. From a starting position (position 1), guide pin 210 moves along an increment ramp 520 (position 2) into a race 510 (position 3) when the shuttle 200 is moved toward the piston 300 to cut the tissue sample X. When the shuttle 200 is moved away from piston 300, the guide pin 210 moves along a race 510 toward an increment ramp 520 and the device is reset (position 4). Accordingly, the piston 300 may move toward the platform 100 incrementally as the shuttle 200 is moved back and forth horizontally along the Z-axis in two motions. Furthermore, in some embodiments, the device 1 maintains a downward force on the piston 300 either through biasing members 120 and/or with the application of a downward force by a user of the device 1. In embodiments in which the device 1 maintains a downward force on the piston 300 through one or more biasing members 120, from the starting position (position 1) shown in FIG. 4A the movement of the shuttle 200 toward the piston 300 actuates the biasing member 120, which urges the piston 300 to move downwards towards the platform 100 and the guide pin 210 to trace a path relative to raceway 500 that proceeds along an increment ramp 520 (position 2) into a race 510 (position 3) to cut the tissue sample X in both the horizontal (i.e., the Z-axis) and vertical (i.e., the X-axis) directions. In some embodiments, the guide pin(s) 210 may be moved into the outer region 530 absent of guides 540 and ribs 512 and the guide pin(s) 210 may bypass the races 510 and increment ramps 520, allowing for the piston 300 to be moved to a desired position in the vertical direction (i.e., the X-axis). This outer region 530 absent of guides 540 and ribs 512 allows for the piston 300 to be moved up and down to a desired race 510 quickly, without moving the guide pin(s) 210 through all the races 510 and increments ramps 520. Because the outer region 530 absent of guides 540 and ribs 512 provides a space where the guide pin(s) 210 are unbound, when the guide pin(s) 210 are moved into outer region 530 absent of guides 540 and ribs 512, the piston 300 is free to move in the vertical direction (i.e., the X-axis).

In further embodiments, device 1 may have the configuration as shown in FIGS. 3A-2 and 3B-2. For embodiments shown FIGS. 3A-2 and 3B-2, raceway 500 includes races or channels 510 that communicate with shuttle 200 via guide pin(s) 210. Races 510 may also include guides 540 and increment ramps 520 at both ends of races 510. Communication between guide pin(s) 210, races 510, and increment ramps 520 allow for the piston 300 to progress toward the platform 100 (e.g., in direction along the X-axis) each time the shuttle 200 slides in either direction horizontally along the Z-axis. For embodiments shown in FIGS. 3A-2 and 3B-2, the relationship between shuttle 200 and raceway 500 is depicted in greater detail in FIG. 4B when slicing a tissue sample X. For embodiments shown FIGS. 3A-2 and 3B-2, the raceway 500 includes an outer region 530 absent of guides 540 and ribs 512 on both ends, 502 and 503, of the raceway 500. This outer region 530 absent of guides 540 and ribs 512 allows for the piston 300 to be moved up and down to a desired race 510 quickly, without moving the guide pin(s) 210 through all the races 510 and increments ramps 520. Because the outer region 530 absent of guides 540 and ribs 512 provides a space where the guide pin(s) 210 are unbound, when the guide pin(s) 210 are moved into outer region 530, the piston 300 is free to move in the vertical direction (i.e., the X-axis).

For embodiments shown in FIGS. 3A-2 and 3B-2, the shuttle 200 may be moved through five positions (i.e., positions, 1-5) in the raceway 500, as shown in FIG. 4B. From a starting position (position 1), guide pin 210 moves along an increment ramp 520 (position 2) to one end of a race 510 towards the other end of the race 510 (position 3) when the shuttle 200 is moved toward the piston 300 to cut the tissue sample X. When the shuttle 200 is next moved in the reverse direction along the Z-axis, the guide pin 210 moves along an increment ramp 520 (position 4) to one end of an adjoining race 510 towards the other end of the adjoining race 510 and the device is reset (position 5). Accordingly, the piston 300 may move toward the platform 100 incrementally each time the shuttle 200 is moved in either direction horizontally along the Z-axis. Furthermore, in some embodiments, the device 1 maintains a downward force on the piston 300 either through biasing members 120 and/or with the application of a downward force by a user of the device 1. In embodiments in which the device 1 maintains a downward force (e.g., in the X-direction) on the piston 300 through one or more biasing members 120, from the starting position (position 1) shown in FIG. 4B (i) the movement of the shuttle 200 toward the piston 300 actuates the biasing member 120, which urges the piston 300 to move downwards towards the platform 100 and the guide pin 210 to trace a path relative to raceway 500 that proceeds along an increment ramp 520 (position 2) to one end of a race 510 towards the other end of the race 510 in a first direction (position 3) to cut the tissue sample X and (ii) from which position (position 3) the movement of the shuttle 200 in the reverse direction actuates the biasing member 120, which urges the piston 300 to move downwards towards the platform 100 and the guide pin 210 to trace a path relative to raceway 500 that proceeds along an increment ramp 520 (position 4) to one end of an adjoining race 510 towards the other end of the adjoining race 510 (position 5) to cut the tissue sample X in the horizontal (i.e., the Z-axis) and vertical direction (i.e., the X-axis). In some embodiments, the guide pin(s) 210 may be moved into the outer region 530 absent of guides 540 and ribs 512 and the piston 300 may bypass the races 510 and increment ramps 520 and moved to a desired position in the vertical direction (i.e., the X-axis). As described previously, in some embodiments first and second edges 231 and 232 may each be a cutting edge. Such a configuration allows blade 230 to horizontally cut the tissue sample X as shuttle 200 is moved in both the first direction (position 3) and the reverse direction (position 5).

In some embodiments, the distance between each race 510 of the raceway 500 is about 0.5 mm to about 10 mm. In some embodiments, the distance between each race 510 of the raceway 500 is about 1 mm to about 5 mm. In some embodiments, the distance between each race 510 of the raceway 500 is about 3 mm. In some embodiments, the distance between each race 510 of the raceway 500 is about 0.5 mm to about 10 mm, which translates to a tissue sample slice having a thickness of about 0.5 mm to about 10 mm. In some embodiments, the distance between each race 510 of the raceway 500 is about 1 mm to about 5 mm, which translates to a tissue sample slice having a thickness of about 1 mm to about 5 mm. In some embodiments, the distance between each race 510 of the raceway 500 is about 3 mm, which translates to a tissue sample slice having a thickness of about 3 mm. In some embodiments, the distance between each race 510 of the raceway 500 is about 2 mm, which translates to a tissue sample slice having a thickness of about 2 mm. In some embodiments, the distance between each race 510 of the raceway 500 is about 1 mm, which translates to a tissue sample slice having a thickness of about 1 mm.

In some embodiments, the distance between each race 510 of the raceway 500 is about 1 mm to about 5 mm, which translates to a tissue sample slice having a thickness of about 1 mm to about 5 mm. In some embodiments, the distance between each race 510 of the raceway 500 is about 1 mm to about 4 mm, which translates to a tissue sample slice having a thickness of about 1 mm to about 4 mm. In some embodiments, the distance between each race 510 of the raceway 500 is about 1 mm to about 3 mm, which translates to a tissue sample slice having a thickness of about 1 mm to about 3 mm. In some embodiments, the distance between each race 510 of the raceway 500 is about 1 mm to about 2 mm, which translates to a tissue sample slice having a thickness of about 1 mm to about 2 mm. In some embodiments, the distance between each race 510 of the raceway 500 is about 0.5 mm to about 3.5 mm, which translates to a tissue sample slice having a thickness of about 0.5 mm to about 3.5 mm. In some embodiments, the distance between each race 510 of the raceway 500 is about 0.5 mm to about 3.0 mm, which translates to a tissue sample slice having a thickness of about 0.5 mm to about 3.0 mm. In some embodiments, the distance between each race 510 of the raceway 500 is about 0.5 mm to about 2.5 mm, which translates to a tissue sample slice having a thickness of about 0.5 mm to about 2.5 mm. In some embodiments, the distance between each race 510 of the raceway 500 is about 0.5 mm to about 2.0 mm, which translates to a tissue sample slice having a thickness of about 0.5 mm to about 2.0 mm. In some embodiments, the distance between each race 510 of the raceway 500 is about 0.5 mm to about 1.5 mm, which translates to a tissue sample slice having a thickness of about 0.5 mm to about 1.5 mm. In some embodiments, the distance between each race 510 of the raceway 500 is about 0.5 mm to about 1.0 mm, which translates to a tissue sample slice having a thickness of about 0.5 mm to about 1.0 mm.

In some embodiments, the distance between each race 510 of the raceway 500 is less than 0.5, 0.6, 0.7, 0.8, 0.9, 1, 2, 3, 4, 5, 6, 7, 8, 9, or 10 mm. In some embodiments, the distance between each race 510 of the raceway 500 is greater than 0.5, 0.6, 0.7, 0.8, 0.9, 1, 2, 3, 4, 5, 6, 7, 8, 9, or 10 mm. In some embodiments, the distance between each race 510 of the raceway 500 is about 0.5, 0.6, 0.7, 0.8, 0.9, 1, 2, 3, 4, 5, 6, 7, 8, 9, or 10 mm.

In some embodiments, the distance between each race 510 of the raceway 500 is less than 0.5, 0.6, 0.7, 0.8, 0.9, 1, 2, 3, 4, 5, 6, 7, 8, 9, or 10 mm, which translates to a tissue sample slice having a thickness of less than 0.5, 0.6, 0.7, 0.8, 0.9, 1, 2, 3, 4, 5, 6, 7, 8, 9, or 10 mm. In some embodiments, the distance between each race 510 of the raceway 500 is greater than 0.5, 0.6, 0.7, 0.8, 0.9, 1, 2, 3, 4, 5, 6, 7, 8, 9, or 10 mm, which translates to a tissue sample slice having a thickness of greater than 0.5, 0.6, 0.7, 0.8, 0.9, 1, 2, 3, 4, 5, 6, 7, 8, 9, or 10 mm. In some embodiments, the distance between each race 510 of the raceway 500 is about 0.5, 0.6, 0.7, 0.8, 0.9, 1, 2, 3, 4, 5, 6, 7, 8, 9, or 10 mm, which translates to a tissue sample slice having a thickness of about 0.5, 0.6, 0.7, 0.8, 0.9, 1, 2, 3, 4, 5, 6, 7, 8, 9, or 10 mm.

In some embodiments, the distance between each race 510 of the raceway 500 is less than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each race 510 of the raceway 500 is greater than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each race 510 of the raceway 500 is about 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm.

In some embodiments, the distance between each race 510 of the raceway 500 is less than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, which translates to a tissue sample slice having a thickness of less than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each race 510 of the raceway 500 is greater than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, which translates to a tissue sample slice having a thickness of greater than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each race 510 of the raceway 500 is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, which translates to a tissue sample slice having a thickness of about 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm.

Platform 100 includes a blade array 400, which may be a vertical blade array. As described above, the piston 300 may be biased toward the platform 100 when biasing member 120 is connected to biasing member mounts 320. In some embodiments, biasing the piston 300 toward the platform 100 includes biasing the tissue mount 310 of the piston 300 toward the blade array 400. In some embodiments, this biasing provides a downward force (in a direction along the X-axis) to push tissue sample X through the blade array 400.

The tissue mount 310 is configured to maintain the position of the tissue sample in the device 1 during operation. In some embodiments, tissue mount 310 may include a plurality of protrusions 311 that may assist in maintaining the positon of the tissue sample in the device and at the blade array 400. In some embodiments, the plurality of protrusions 311 may resemble oblong “fingers” that are sized to push the tissue sample X through the blade array 400.

In some embodiments, the blade array 400 includes a plurality of blade slits 410 that are configured to hold and retain blades 411, which are assembled in a crosswise pattern in opening 420. In some embodiments, the blades 411 may be, for example, metal or ceramic blades. In some embodiments, the blades 411 may be replaced with wires held in place at blade slits 410. The wires may be, for example, stainless steel wires that are thin enough to cut the tissue sample as the tissue sample is pushed through blade array 400 by piston 300. In some embodiments, the wires of blade array 400 are woven to form a grid or mesh. In other embodiments, each wire is independently positioned with respect to the other wires. In some embodiments, the blade array 400 (and/or opening 420) is about 40 mm×40 mm, for example. In some embodiments the blade slits 410 in blade array 400 will be completely filled with blades 411 or wires. In some embodiments the blade slits 410 in blade array 400 need not be completely filled with blades 411 or wires. In some embodiments, the area of blade array 400 (and/or opening 420) may be customizable based on the size of the tissue sample to be cut.

In some embodiments, the shuttle blade 230 is positioned in the shuttle 200 to contact the blade array 400 upon movement of the shuttle 200 along the Z-axis. In some embodiments, blade 230 is configured to move at a predetermined distance below blade array 400 (e.g., from 0.5 mm to 10 mm) as shuttle 200 is moved back and forth. During operation of such embodiments, as the piston 300 advances toward the platform 100, the tissue sample is pressed into the opening 420 and cuts are first made along a vertical axis (i.e., the X-axis), before the shuttle 200 with shuttle blade 230 delivers a horizontal cut along the Z-axis, to provide a plurality of tissue fragments from the tissue sample.

In some embodiments, the shuttle blade 230 is positioned in the shuttle 200 at a distance above the blade array 400 equal to the distance between each race 510 from any adjoining race 510 in the race array 500. During operation of such embodiments, the shuttle 200 with shuttle blade 230 delivers a first horizontal cut along the Z-axis, to provide a slice of tissue, before the piston 300 advances toward the platform 100, pressing the slice of tissue through the blade array 400 to make cuts along a vertical axis (i.e., the X-axis) to provide a plurality of tissue fragments from the tissue sample.

In some embodiments, the blades 411 (blade slits 410) are spaced to provide a plurality of square or rectangular cuts to the tissue sample. In some embodiments, the blades 411 (blade slits 410) are spaced to provide a plurality of square cuts to the tissue sample. In some embodiments, the distance between each blade 411 of the blade array 400 is about 0.5 mm to about 10 mm. In some embodiments, the distance between each blade 411 of the blade array 400 is about 1 mm to about 5 mm. In some embodiments, the distance between each blade 411 of the blade array 400 is about 3 mm. In some embodiments, the distance between each blade 411 of the blade array 400 is about 0.5 mm to about 10 mm, which translates to a tissue sample slice having a length and/or width of about 0.5 mm to about 10 mm. In some embodiments, the distance between each blade 411 of the blade array 400 is about 1 mm to about 5 mm, which translates to a tissue sample slice having a length and/or width of about 1 mm to about 5 mm. In some embodiments, the distance between each blade 411 of the blade array 400 is about 3 mm, which translates to a tissue sample slice having a length and/or width of about 3 mm. In some embodiments, the distance between each blade 411 of the blade array 400 is about 1 mm, which translates to a tissue sample slice having a length and/or width of about 1 mm. In some embodiments, the distance between each blade 411 of the blade array 400 is about 2 mm, which translates to a tissue sample slice having a length and/or width of about 2 mm.

In some embodiments, the distance between each blade 411 of the blade array 400 is less than 0.5, 0.6, 0.7, 0.8, 0.9, 1, 2, 3, 4, 5, 6, 7, 8, 9, or 10 mm. In some embodiments, the distance between each blade 411 of the blade array 400 is greater than 0.5, 0.6, 0.7, 0.8, 0.9, 1, 2, 3, 4, 5, 6, 7, 8, 9, or 10 mm. In some embodiments, the distance between each blade 411 of the blade array 400 is about 0.5, 0.6, 0.7, 0.8, 0.9, 1, 2, 3, 4, 5, 6, 7, 8, 9, or 10 mm.

In some embodiments, the distance between each blade 411 of the blade array 400 is less than 0.5, 0.6, 0.7, 0.8, 0.9, 1, 2, 3, 4, 5, 6, 7, 8, 9, or 10 mm, which translates to a tissue sample slice having a length and/or width of less than 0.5, 0.6, 0.7, 0.8, 0.9, 1, 2, 3, 4, 5, 6, 7, 8, 9, or 10 mm. In some embodiments, the distance between each blade 411 of the blade array 400 is greater than 0.5, 0.6, 0.7, 0.8, 0.9, 1, 2, 3, 4, 5, 6, 7, 8, 9, or 10 mm, which translates to a tissue sample slice having a length and/or width of greater than 0.5, 0.6, 0.7, 0.8, 0.9, 1, 2, 3, 4, 5, 6, 7, 8, 9, or 10 mm. In some embodiments, the distance between each blade 411 of the blade array 400 is about 0.5, 0.6, 0.7, 0.8, 0.9, 1, 2, 3, 4, 5, 6, 7, 8, 9, or 10 mm, which translates to a tissue sample slice having a length and/or width of about 0.5, 0.6, 0.7, 0.8, 0.9, 1, 2, 3, 4, 5, 6, 7, 8, 9, or 10 mm.

In some embodiments, the distance between each blade 411 of the blade array 400 is less than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each blade 411 of the blade array 400 is greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each blade 411 of the blade array 400 is about 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm.

In some embodiments, the distance between each blade 411 of the blade array 400 is less than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, which translates to a tissue sample slice having a length and/or width of less than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each blade 411 of the blade array 400 is greater than 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, which translates to a tissue sample slice having a length and/or width of greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the distance between each blade 411 of the blade array 400 is about 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm, which translates to a tissue sample slice having a length and/or width of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm.

In some embodiments, operation of the device 1 provides tissue fragments from the tissue sample being fragmented of uniform shape. In some embodiments, the resulting tissue fragments will be cubed. In some embodiments, the length, width, and/or thickness of each tissue fragment is about 0.5 mm to about 10 mm. In some embodiments, the length, width, and/or thickness of each tissue fragment is about 1 mm to about 5 mm. In some embodiments, the length, width, and/or thickness of each tissue fragment is about 3 mm. In some embodiments, the length, width, and/or thickness of each tissue fragment is less than 0.5, 0.6, 0.7, 0.8, 0.9, 1, 2, 3, 4, 5, 6, 7, 8, 9, or 10 mm. In some embodiments, the length, width, and/or thickness of each tissue fragment is greater than 0.5, 0.6, 0.7, 0.8, 0.9, 1, 2, 3, 4, 5, 6, 7, 8, 9, or 10 mm. In some embodiments, the length, width, and/or thickness of each tissue fragment is about 0.5, 0.6, 0.7, 0.8, 0.9, 1, 2, 3, 4, 5, 6, 7, 8, 9, or 10 mm.

In some embodiments, the resulting tissue fragments will be cubed. In some embodiments, the length, width, and thickness of each tissue fragment is about 0.5 mm to about 1 mm. In some embodiments, the length, width, and thickness of each tissue fragment is about 1 mm to about 1.5 mm. In some embodiments, the length, width, and thickness of each tissue fragment is about 1 mm to about 1.1 mm. In some embodiments, the length, width, and thickness of each tissue fragment is about 1.1 mm to about 1.2 mm. In some embodiments, the length, width, and thickness of each tissue fragment is about 1.2 mm to about 1.3 mm. In some embodiments, the length, width, and thickness of each tissue fragment is about 1.3 mm to about 1.4 mm. In some embodiments, the length, width, and thickness of each tissue fragment is about 1.4 mm to about 1.5 mm. In some embodiments, the length, width, and thickness of each tissue fragment is about 1 mm. In some embodiments, the length, width, and thickness of each tissue fragment is about 1.1 mm. In some embodiments, the length, width, and thickness of each tissue fragment is about 1.2 mm. In some embodiments, the length, width, and thickness of each tissue fragment is about 1.3 mm. In some embodiments, the length, width, and thickness of each tissue fragment is about 1.4 mm. In some embodiments, the length, width, and thickness of each tissue fragment is about 1.5 mm.

In some embodiments, the length, width, and thickness of each tissue fragment is less than 0.5, 0.6, 0.7, 0.8, 0.9, 1, 2, 3, 4, 5, 6, 7, 8, 9, or 10 mm. In some embodiments, the length, width, and thickness of each tissue fragment is greater than 0.5, 0.6, 0.7, 0.8, 0.9, 1, 2, 3, 4, 5, 6, 7, 8, 9, or 10 mm. In some embodiments, the length, width, and thickness of each tissue fragment is about 0.5, 0.6, 0.7, 0.8, 0.9, 1, 2, 3, 4, 5, 6, 7, 8, 9, or 10 mm.

In some embodiments, the length, width, and thickness of each tissue fragment is less than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the length, width, and thickness of each tissue fragment is greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm. In some embodiments, the length, width, and thickness of each tissue fragment is about 0.1, 0.2, 0.3, 0.4, 0.5, 0.6, 0.7, 0.8, 0.9, 1.0, 1, 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm.

In some embodiments, the length, width, and thickness of each tissue fragment is less than 1.0, 1.1, 1.2, 1.3, 1.4, or 1.5 mm. In some embodiments, the length, width, and thickness of each tissue fragment is greater than 1.0, 1.1, 1.2, 1.3, 1.4, or 1.5 mm. In some embodiments, the length, width, and thickness of each tissue fragment is about 1.0, 1.1, 1.2, 1.3, 1.4, or 1.5 mm.

In an embodiment, a user may operate a device 1 described herein by moving the shuttle 200 into position 1 (FIGS. 4A-4B) and loading a tissue sample at the blade array 400. The piston 300 may then be advanced by, for example, moving the shuttle 200 back and forth so that the tissue mount 310 may abut a surface of the tissue sample. Alternatively, the shuttle 200 may be withdrawn such that guide pin(s) 210 are removed from the raceway 500 and tissue mount 310 may then be lowered to abut a surface of the tissue sample X. With the tissue sample in position between the tissue mount 310 and the blade array 400, the shuttle 200 may then be moved back and forth along the horizontal axis to bias the tissue mount 310 against the tissue sample and thereby press the tissue sample X through the blade array 400 to provide vertical cuts to the tissue sample X. After the tissue sample X is pressed through the blade array 400, the shuttle blade 230 of the shuttle 200 then provides a horizontal cut to the tissue sample to provide a plurality of tissue fragments or pieces. The plurality of tissue fragments or pieces may then be collected and stored for further use. In some embodiments the tissue fragments or pieces may then be placed in a cell culturing device or a cell culturing container for further processing. Some embodiments of further processing are described below.

In some embodiments, the tissue sample X is a tumor sample that is obtained from a subject (e.g., a human or animal subject). In some embodiments, device 1 may be particularly useful for cutting the tumor sample into smaller fragments for the purpose of culturing cells obtained from the tumor sample. The cells, for example, may include tumor infiltrating lymphocytes (TILs) which may be further cultured, expanded, and utilized for immunotherapy according to certain embodiments. In some embodiments, tumor fragments that were cut by device 1 may be positioned into one or more tissue culture devices (e.g., culture dishes, gas-permeable flasks, gas-permeable culture bags, or other gas-permeable containers) along with a suitable culture medium and cultured for a predetermined period of time in order to expand a first population of TILs from the tumor sample fragments into a second, larger population of TILs. In some embodiments, the culture medium may be supplemented with, for example, IL-2, OKT-3 and/or antigen presenting cells (APCs). In some embodiments, each fragment is positioned into a separate tissue culture device. In other embodiments, a plurality of fragments may be cultured in the same tissue culture device. The predetermined period of time may be, for example, from 1 day to 28 days, from 1 day to 8 days, from 3 days to 11 days, from 3 days to 14 days, from 7 days to 11 days, from 7 days to 14 days, from 11 days to 22 days, etc.

In some embodiments, the second population of TILs may be further expanded into a third population of TILs. In some embodiments, a second expansion is performed by supplementing the cell culture medium with additional IL-2, OKT-3 and/or APCs and culturing the TILs for a second predetermined period of time (e.g., from 1 day to 28 days, from 1 day to 8 days, from 3 days to 11 days, from 3 days to 14 days, from 7 days to 11 days, from 7 days to 14 days, from 11 days to 22 days, etc.). In some embodiments, the second population of TILs may be filtered to remove debris and transferred to new tissue culture devices (e.g., culture dishes, gas-permeable flasks, gas-permeable culture bags, or other gas-permeable containers) for the second expansion. The third population of TILs following the second expansion may then be harvested for use in administration to the subject or can be cryopreserved. In some embodiments, the process of cutting the tumor sample with device 1, and culturing/expanding the TILs to obtain the second and third populations of TILs may occur for example, over a total period of about 14 days to about 42 days, e.g., about 22 days to about 28 days. In some embodiments, the cutting of the tumor sample with device 1 and the culturing/expanding of the TILs may occur in a closed system. “Closed system” refers to a system that is closed to the outside environment. For example, once a tumor sample is introduced into the closed system, the system is not opened to the outside environment until the final population of TILs have been harvested. Transfer of the tissue fragments obtained from device 1 to the tissue culture device(s) and/or transfer of the TILs from one tissue culture device to another may occur, for example, via closed tubing between the components that is sealed from the outside environment. In some embodiments, device 1 and/or the tissue culture devices may be enclosed in an incubator configured to maintain these components at a predetermined temperature (e.g., about 37° C.) and/or atmospheric condition (e.g., 5% CO2).

In some embodiments, the TILs are optionally genetically engineered or modified. For example, the TILs may be genetically engineered or modified to include additional functionalities, including, but not limited to, a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19).

In some embodiments, the tissue fragments produced by cutting tissue sample X with device 1 may be stored for a period of time prior to further processing. In some embodiments, the tissue fragmens may be stored in a suitable aqueous solution, for example, a buffered saline solution or culture medium before being positioned in the one or more tissue culture devices. In some embodiments, the tissue fragments produced by cutting tissue sample X with device 1 may be cryopreserved after cutting by device 1. For example, the cut tissue fragments may be placed in a suitable container along with a cryopreservation medium and frozen in a freezer (e.g., at or below −130° C., for example, at about −150° C. to about −196° C.). When appropriate, the frozen tissue fragments are removed from the freezer and thawed, for example, in a 37° C. water bath. The thawed tissue fragments may then be placed in tissue culture devices and cultured/expanded as described, for example, in the embodiments above.

In some embodiments, the shuttle 200 may be connected to a motor, such as a reciprocating motor, and optionally an actuator, to allow for the automated movement of the shuttle 200. In some embodiments, the process of cutting tissue sample X is fully automated. In some embodiments, the process of cutting tissue sample X is semi-automatic, or operated partly automatically and partly by hand. In a semi-automatic embodiment, for example, the action mechanism of device 1 may require an operator to load and position the tissue sample in device 1, but will utilize the motor to move the shuttle 200 and cut tissue sample X in the horizontal direction (Z-axis). In some embodiments, the motor can be disconnected. An advantage of a disconnectable motor includes being able to manually use the machine in the event of mechanical or electrical issues. A disconnectable motor also allows for either the device or the motor to be serviced more easily in the case of a malfunction.

In some embodiments, the any part of the device 1 (e.g., the shuttle 200, platform 100, and/or piston 300) may be prepared from a material that is cleanable, sterilizable, and/or does not affect cell function. In some embodiments, the any part of the device 1 (e.g., the shuttle 200, platform 100, and/or piston 300) may be prepared from passivated stainless steel.

In some embodiments, device 1 as a whole, or any of the components thereof (e.g., the shuttle 200, platform 100, and/or piston 300) may be sterilized by autoclave.

While certain embodiments of the described disclosure have been described and/or exemplified above, various other embodiments will be apparent to those skilled in the art from the foregoing disclosure. The described disclosure is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed disclosure. All devices and methods described herein that embody the described disclosure can, in alternate embodiments, be more specifically defined by any of the transitional terms “comprising,” “consisting essentially of,” and “consisting of.”

We claim:

1. A device for preparing a plurality of tissue fragments from a tissue sample, comprising:
a platform having a central opening;
a shuttle slidably coupled to the platform and including a shuttle blade;
a plurality of cutting elements mounted across the central opening of the platform and comprising a plurality of blades each generally perpendicular to the shuttle blade; and
a piston slidably coupled to the platform and slidably coupled to the shuttle along a predefined raceway and configured to urge the tissue sample into the blade array.

2. The device of claim 1, wherein the platform comprises a channel and the shuttle includes a projection slidably coupled to the platform through the channel.

3. The device of claim 1, wherein the raceway comprises a plurality of races.

4. The device of claim 3, wherein each race of the plurality of races is coupled to an adjacent race by an extending increment ramp.

5. The device of claim 3 or 4, wherein each race of the plurality of races is separated by about 0.5 mm to about 10 mm.

6. The device of claim 5, wherein each race of the plurality of races is separated by about 1 mm to about 5 mm.

7. The device of claim 6, wherein each race of the plurality of races is separated by about 3 mm.

8. The device of claim 1, wherein the plurality of tissue fragments each have a thickness of about 0.5 mm to about 10 mm.

9. The device of claim 8, wherein the plurality of tissue fragments each have a thickness of about 1 mm to about 5 mm.

10. The device of claim 9, wherein the plurality of tissue fragments each have a thickness of about 3 mm.

11. The device of any one of the preceding claims, wherein the shuttle comprises a guide pin slideably received in the raceway.

12. The device of any one of the preceding claims, wherein moving the shuttle through the raceway moves the shuttle blade and then the blade array through a bottom portion of the tissue sample.

13. The device of any one of claims 1 to 11, wherein moving the shuttle through the raceway moves the blade array and then the shuttle blade through a bottom portion of the tissue sample.

14. The device of any one of the preceding claims, further comprising a motor configured to move the shuttle relative to the platform as defined by the shape of the raceway.

15. The device of any one of the preceding claims, wherein the raceway comprises at least two raceways.

16. The device of any one of the preceding claims, wherein the device further comprises a biasing member disposed between the piston and the platform, and wherein the biasing member biases the piston to move towards the platform.

17. The device of claim 16, wherein movement of the shuttle towards the piston actuates the biasing member to move the piston towards the platform to urge the tissue sample into the blade array.

18. The device of claim 17, wherein actuation of the biasing member moves a guide pin in the shuttle slideably received in a race in the raceway up an extending ramp into an adjoining race in the raceway, thereby moving the piston towards the platform to urge the tissue sample into the blade array.

19. The device of any one of claims 1-18, wherein the shuttle blade is double-edged.

20. The device of claim 19, wherein each edge is angled or has a triangular shape, each edge is rectangular or is straight, each edge has a curved shape, or each edge has a crescent shape.

21. The device of any one of claims 1 to 20, wherein the plurality of cutting elements mounted across the central opening is an array of metal blades.

22. The device of any one of claims 1 to 20, wherein the plurality of cutting elements mounted across the central opening is an array of wires.

23. The device of any one of claims 16 to 22, wherein the platform comprises a channel with a first end and a second end, wherein the shuttle includes a projection slidably coupled to the platform through the channel, and wherein the movement of the shuttle from a position proximal to the first end or the second end of the channel towards the piston actuates the biasing member to move the piston towards the platform to urge the tissue sample into the blade array.

24. The device of claim 23, wherein actuation of the biasing member moves a guide pin in the shuttle slideably received in a race with a first terminus and a second terminus in the raceway up a first extending ramp adjacent to the first terminus or up a second extending ramp adjacent to the second terminus into an adjoining race in the raceway, thereby moving the piston towards the platform to urge the sample into the blade array.

25. A method of using the device of any one of the preceding claims to cut a tissue sample from a patient into a plurality of tissue fragments, the method comprising loading the tissue sample into a tissue sample mount and moving the shuttle through the raceway to cut the tissue sample in two axes to prepare a plurality of tissue fragments from the tissue sample.

26. The method of claim 25, wherein the plurality of tissue fragments each have a thickness of about 0.5 mm to about 10 mm.

27. The method of claim 26, wherein the plurality of tissue fragments each have a thickness of about 1 mm to about 5 mm.

28. The method of claim 26, wherein the plurality of tissue fragments each have a thickness of about 3 mm.

29. The method of claim 26, wherein the plurality of tissue fragments each have a thickness of about 1 mm.

30. The method of any one of claims 25 to 29, wherein the plurality of tissue fragments each have a width of about 0.5 mm to about 10 mm.

31. The method of any one of claims 25 to 30, wherein the plurality of tissue fragments each have a width of about 1 mm to about 5 mm.

32. The method of any one of claims 25 to 30, wherein the plurality of tissue fragments each have a width of about 3 mm.

33. The method of any one of claims 25 to 30, wherein the plurality of tissue fragments each have a width of about 1 mm.

34. The method of any one of claims 25 to 33, further comprising placing the plurality of tissue fragments into one or more tissue culturing devices.

35. The method of any one of claims 25 to 33, further comprising cryopreserving the plurality of tissue fragments.

36. The method of any one of claims 25 to 33, further comprising storing the plurality of tissue fragments.
